(19) United States
(12) Patent Application Publication
(10) Pub. No.: US 2014/0000618 A1
(43) Pub. Date: Jan. 2, 2014

(54) HELICAL FOREHEAD SUPPORT ADJUSTMENT MECHANISM

(71) Applicant: Jason Anthony Belton, Norton, OH (US)

(72) Inventor: Jason Anthony Belton, Norton, OH (US)

(73) Assignee: KONINKLIJKE PHILIPS N.V., EINDHOVEN (NL)

(21) Appl. No.: 14/005,381

(22) PCT Filed: Mar. 13, 2012
(86) PCT No.: PCT/IB12/51166

§ 371 Date: Sep. 16, 2013

Related U.S. Application Data
(60) Provisional application No. 61/454,786, filed on Mar. 21, 2011.

(51) Int. Cl. A61M 16/06; A61M 16/00

(52) U.S. Cl. CPC A61M 16/0683; A61M 16/0057
USPC 128/205.25

(57) ABSTRACT

A patient interface device (25) includes a patient sealing assembly (35) including a cushion (40) and a frame member (45) coupled thereto, and an adjustable forehead support assembly (100). The adjustable forehead support assembly includes an adjustment mechanism (102) coupled to a forehead cushion (104), the adjustment mechanism including a forehead cushion support member (108) having a base portion (114) coupled to the forehead cushion, a projecting member (116) extending from the base portion, a rotatable knob member (110) having a surface (128) having a helical groove (130) provided therein, and a post (134) coupled to one of the frame member and the projecting member. The post is received within the helical groove, and rotation of the knob member causes a position of the post within the helical grove to change thereby causing relative movement between the projecting member and the frame member and movement of the frame member and the cushion relative to the forehead cushion.

### CROSS-REFERENCE TO RELATED APPLICATIONS

This patent application claims the priority benefit under 35 U.S.C. §119(e) of U.S. Provisional Application No. 61/454,786 filed on Mar. 21, 2011, the contents of which are herein incorporated by reference.

### BACKGROUND OF THE INVENTION

1. Field of the Invention

The present invention relates to patient interface devices for transporting a gas to and/or from an airway of a user, and in particular, to a patient interface device including a mechanism for adjusting a forehead support of the patient interface device that employs a helical adjustment mechanism.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), or congestive heart failure.

Non-invasive ventilation and pressure support therapies involve the placement of a patient interface device including a mask component on the face of a patient. The mask component may be, without limitation, a nasal mask that covers the patient's nose, a nasal cushion having nasal prongs that are received within the patient's nares, a nasal/oral mask that covers the nose and mouth, or a full face mask that covers the patient's face. The patient interface device interfaces the ventilator or pressure support device with the airway of the patient, so that a flow of breathing gas can be delivered from the pressure/flow generating device to the airway of the patient. It is known to maintain such devices on the face of a wearer by a headgear having one or more straps adapted to fit over/around the patient's head.

Because such patient interface devices are typically worn for an extended period of time, it is important for the headgear to maintain the mask component of the device in a tight enough seal against the patient's face without discomfort. One area where fit and comfort is often a concern is the bridge of the patient's nose, as most patient interface devices will apply a pressure to this area. If this pressure is not able to be managed effectively, either or both of a poor fit or patient discomfort will result, thereby limiting the effectiveness of the device.

### SUMMARY OF THE INVENTION

Accordingly, it is an object of the present invention to provide a patient interface device that overcomes the shortcomings of conventional patient interface devices. This object is achieved according to one embodiment of the present invention by providing a patient interface device that includes a selectively adjustable forehead support assembly that allows for simple and convenient management of the nose bridge pressure that is applied by the patient interface device.

In one embodiment, a patient interface device is provided that includes a patient sealing assembly for delivering a flow of breathing gas to an airway of a patient. The patient sealing assembly includes a cushion, a frame member coupled to the cushion, and an adjustable forehead support assembly provided at a distal end of the frame member. The adjustable forehead support assembly includes an adjustment mechanism coupled to a forehead cushion. The adjustment mechanism including a forehead cushion support member having a base portion coupled to the forehead cushion, a projecting member extending from the base portion, a rotatable knob member having a surface having a helical groove provided therein, and a post coupled to one of the frame member and the projecting member. The post is received within the helical groove such that rotation of the knob member causes a position of the post within the helical grove to change thereby causing relative movement between the projecting member and the frame member, and wherein movement of the frame member relative to the projection member causes movement of the frame member and the cushion relative to the forehead cushion.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a side schematic diagrams of a system adapted to provide a regimen of respiratory therapy to a patient according to one exemplary embodiment of the present invention;

FIG. 2 is a side elevational view and FIG. 3 is a top plan view of an exemplary embodiment of an adjustable forehead support assembly forming a part of the patient interface device of the system of FIG. 1;

FIG. 4 is a top plan view of a forehead cushion support member forming a part of the adjustable forehead support assembly of FIGS. 2 and 3;

FIG. 5 is a schematic illustration of a rotatable knob member and a drive beam forming a part of the adjustable forehead support assembly of FIGS. 2 and 3;

FIG. 6 is an isometric view and FIG. 7 is a side elevational view of an adjustable forehead support assembly according to an alternative exemplary embodiment of the present invention;

FIG. 8 is a front isometric view of a frame member forming part of the patient interface device of the system of FIGS. 6 and 7;

FIG. 9 is a front isometric view of a forehead cushion support member forming part of the patient interface device of the system of FIGS. 6 and 7; and

FIGS. 10 and 11 are side elevational and bottom isometric views, respectively, of a knob member forming part of the patient interface device of the system of FIGS. 6 and 7.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

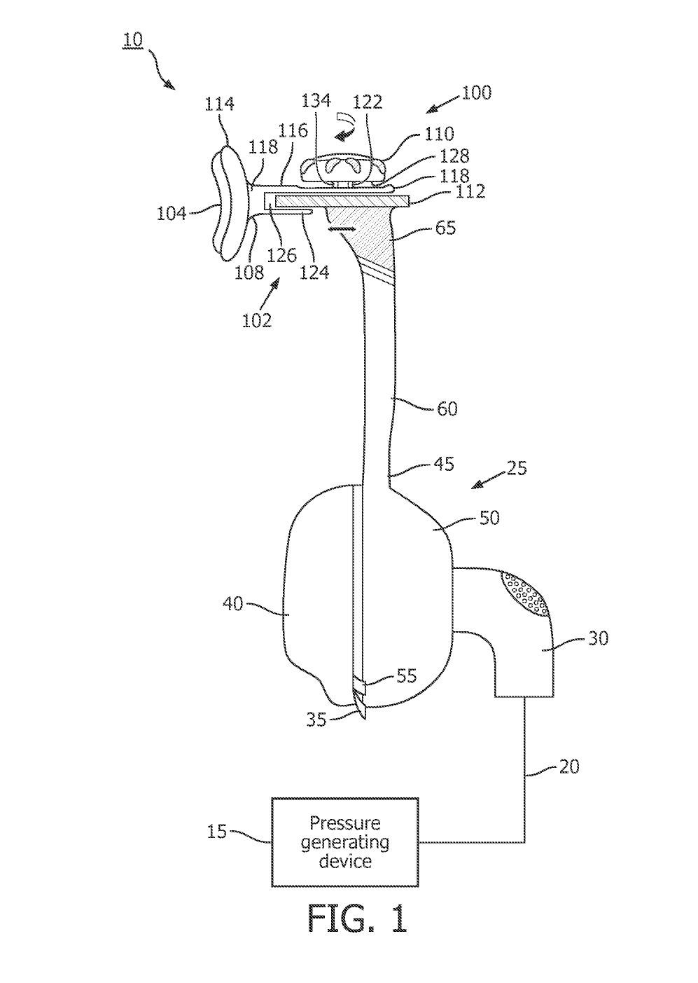

A system 10 adapted to provide a regimen of respiratory therapy to a patient according to one exemplary embodiment is generally shown in FIG. 1. System 10 includes a pressure generating device 15, a delivery conduit 20, and a patient interface device 25 having a fluid coupling conduit 30. Pressure generating device 15 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Delivery conduit 20 is structured to communicate the flow of breathing gas from pressure generating device 15 to patient interface device 25 through fluid coupling conduit 30, which in the illustrated embodiment is an elbow connector. Delivery conduit 20 and patient interface device 25 are often collectively referred to as a patient circuit.

Patient interface device 25 includes a patient sealing assembly 35, which in the illustrated embodiment is a nasal mask. However, other types of patient sealing assemblies, such as, without limitation, a nasal/oral mask or a nasal cushion, which facilitates the delivery of the flow of breathing gas to the airway of a patient may be substituted for patient sealing assembly 35 while remaining within the scope of the present invention. Patient sealing assembly 35 includes a cushion 40 coupled to a frame member 45. In the illustrated embodiment, cushion 40 is defined from a unitary piece of soft, flexible, cushiony, elastomeric material, such as, without limitation, silicone, an appropriately soft thermoplastic elastomer, a closed cell foam, or any combination of such materials. Also in the illustrated embodiment, frame member 45 is made of a rigid or semi-rigid material, such as, without limitation, an injection molded thermoplastic or silicone, and includes a faceplate portion 50 to which cushion 40 is fluidly attached. An opening in faceplate portion 50, to which fluid coupling conduit 30 is coupled, allows the flow of breathing gas from pressure generating device 15 to be communicated to an interior space defined by cushion 40, and then to the airway of a patient. In an alternative embodiment, cushion 40 may be supported by and received through an orifice in frame member 45 so that fluid coupling conduit 30 can be directly connected to cushion 40 rather than to a faceplate portion. In addition, in the exemplary embodiment, faceplate portion 50 includes first and second socket type connecting members 55 for receiving a ball connector of a respective strap of a headgear component (not shown) to secure patient interface device 8 to the patient's head.

Frame member 45 also includes an elongated connecting member 60 having a distal end 65 that is connected to an adjustable forehead support assembly 100 of patient interface device 25. Adjustable forehead support assembly 100 includes an adjustment mechanism 102 that is coupled to a forehead cushion 104, which in the exemplary embodiment is made of a material that is similar to the material of cushion 40. As described in detail herein, adjustment mechanism 100 provides a mechanism for selectively adjusting the force applied to the bridge of the nose of a patient by an apex portion of cushion 40 by varying the position of connecting member 60, and in particular distal end 65 thereof, with respect to forehead cushion 104.

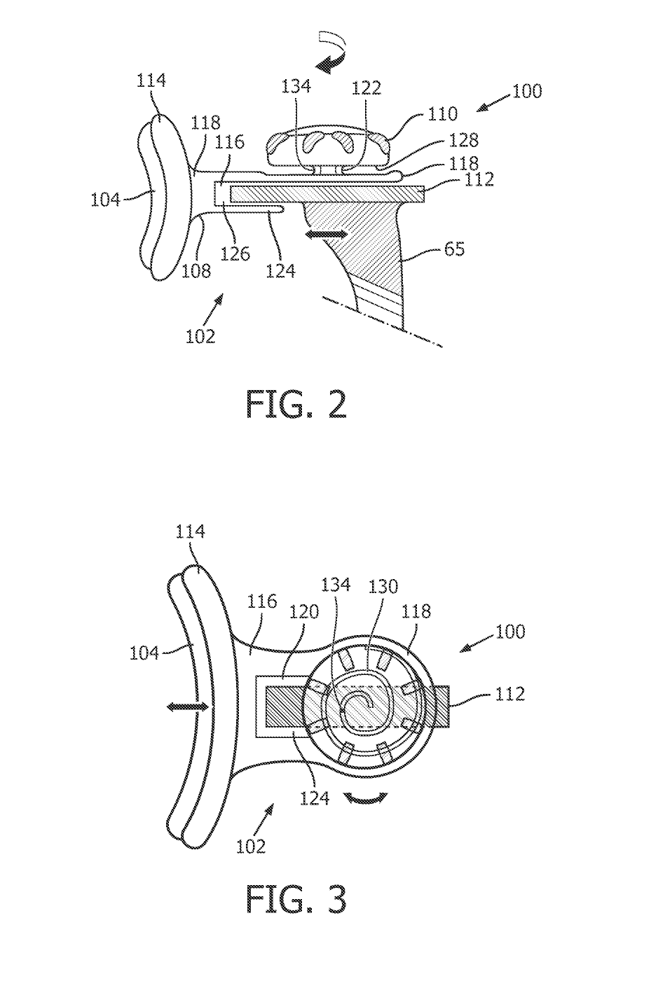

FIG. 2 is a side elevational view and FIG. 3 is a top plan view of adjustable forehead support assembly 100 . As seen in FIGS. 2 and 3, in the illustrated embodiment, adjustment mechanism 102 is in the form of linear translation assembly and is coupled to distal end 65 of a frame member 45. Adjustment mechanism 102 includes a forehead cushion support member 108, a rotatable knob member 110 attached to forehead cushion support member 108, and a drive beam 112 attached to frame member 45.

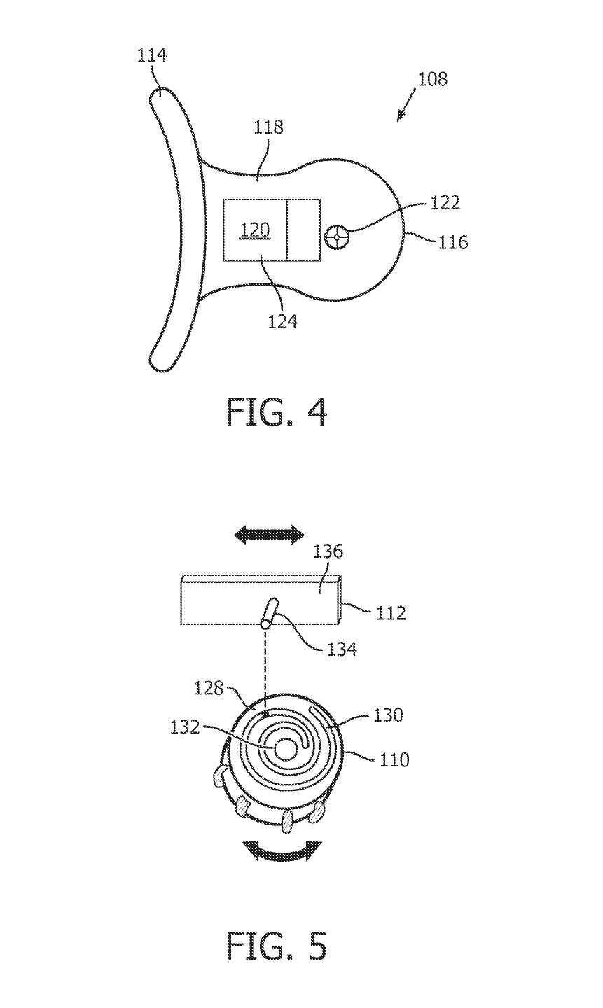

FIG. 4 is a top plan view of forehead cushion support member 108. As seen in FIG. 4, forehead cushion support member 108 includes a base portion 114 to which forehead cushion 104 is attached, and a projecting member 116 extending from base portion 114. Projecting member 116 includes a first support member 118 having an orifice 120 provided therein. In addition, a post 122 is provided on a distal end of first support member 118. Projecting member 116 also includes a second support member 124 (FIG. 2), wherein a gap 126 is provided in between first support member 118 and second support member 124.

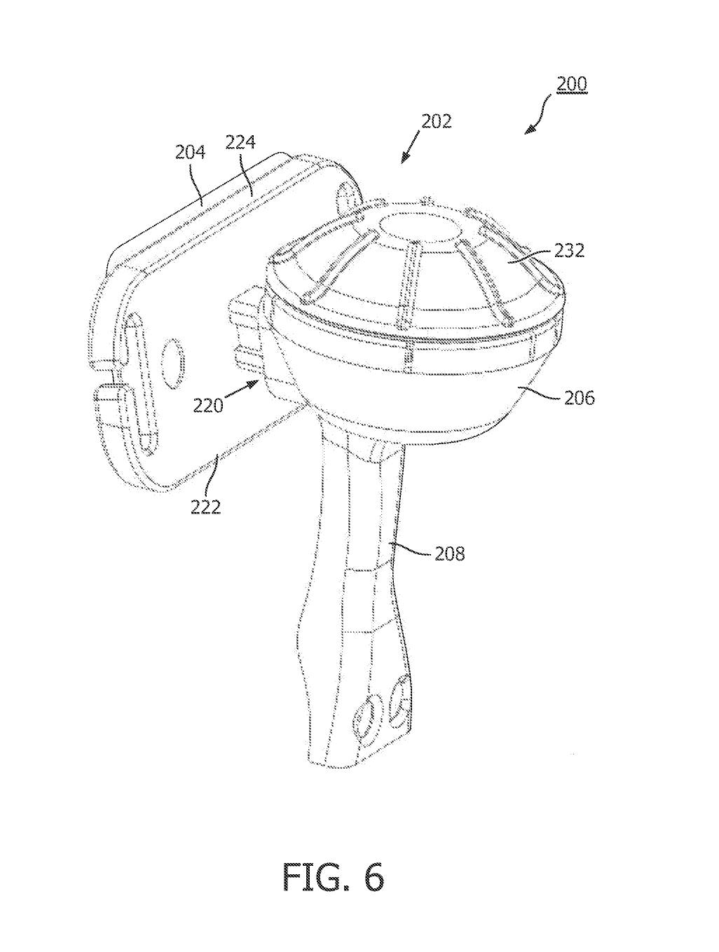

FIG. 5 is a schematic illustration of rotatable knob member 110 and drive beam 112. As seen in FIG. 5, rotatable knob member 110 is in the exemplary embodiment a dome shaped member having a bottom surface 128 having a helical groove 130 and a recess 132 provided therein. Recess 132 is structured to receive post 122 of projecting member 116 in a manner that enables knob member 110 to rotate about post 122. As also seen in FIG. 5, drive beam 112 is an elongated member having a post 134 extending upwardly from a top surface 136 thereof. Post 134 is structured to be received in helical groove 130.

Adjustment mechanism 102 is assembled by inserting the front end of drive beam 112 into gap 126 as seen in FIG. 2. Knob member 110 is then coupled thereto in a manner wherein post 134, which projects through orifice 120, is inserted into helical groove 130 and post 122 is inserted into recess 132. By rotating knob member 110, the position of post 134 along helical groove 130 will be caused to change, which in turn will cause drive beam 112 and thus connecting member 60 frame member 45 to move linearly relative to forehead cushion support member 108 and forehead cushion 104 (which will be at a fixed position on the patient's head). This linear adjustment action allows for selective adjustment of the force that is applied to the bridge of the patient's nose by the apex portion of cushion 40 because varying the position of connecting member 60 as just described will cause the apex portion of cushion 40 to rotate toward and away from the patient's nose.

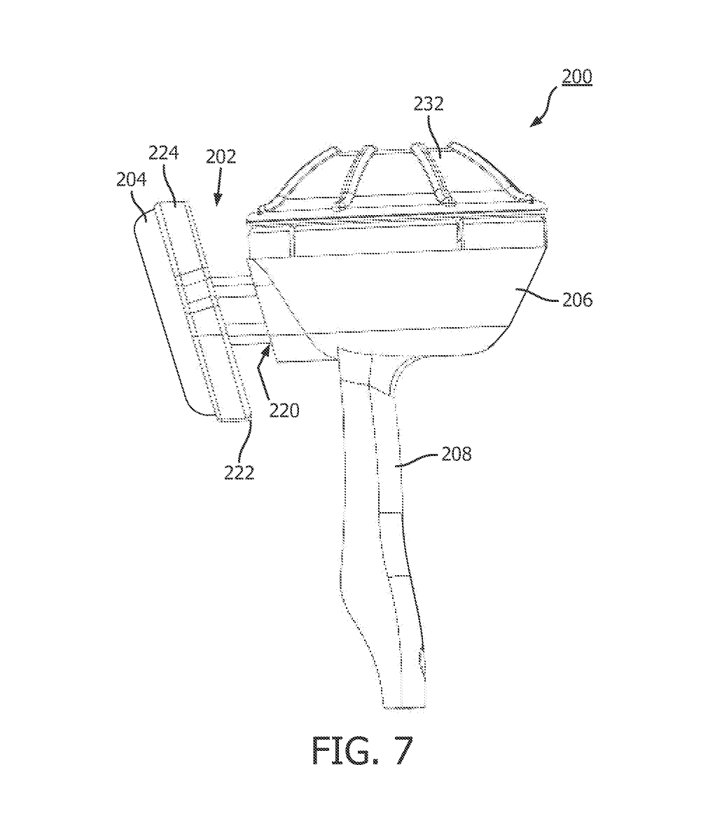

FIG. 6 is an isometric view and FIG. 7 is a side elevational view of an adjustable forehead support assembly 200 according to an alternative embodiment of the present invention. Adjustable forehead support assembly 200 may be substituted for adjustable forehead support assembly 100 of patient interface device 25 in system 10. Adjustable forehead support assembly 200 includes an adjustment mechanism 202 that is coupled to a forehead cushion 204.

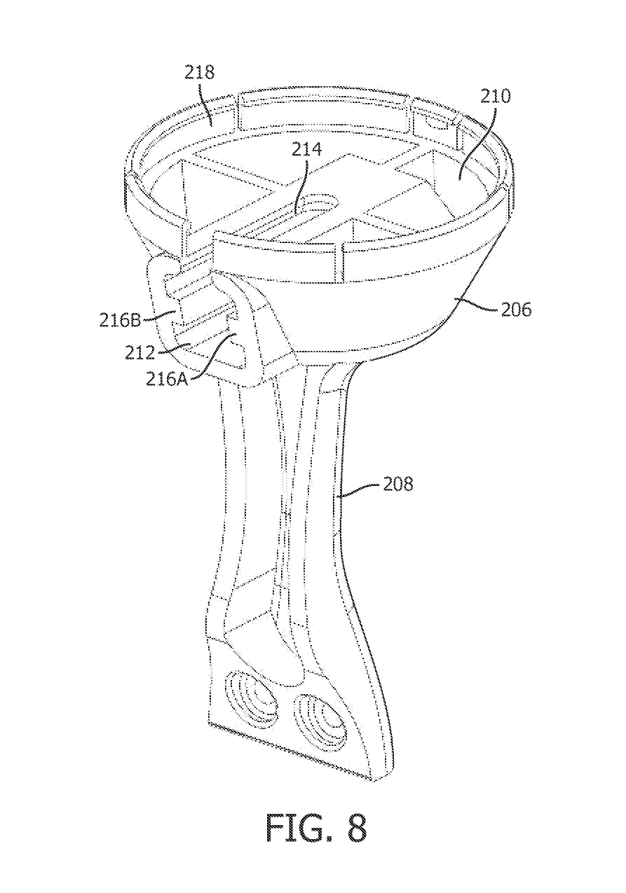

Adjustment mechanism 202 includes a housing portion 206 coupled to the distal end of a frame member 208. FIG. 8 is an isometric view showing housing portion 206 and frame member 208. In the illustrated embodiment, housing portion 206 is formed as an integral part of frame member 208, although in alternative embodiments it may also be formed separately and attached to frame member 208 by a suitable mechanism such as an adhesive.

As seen in FIG. 8, housing portion 206 includes a main chamber 210 having a receiving channel portion 212 formed therein. Receiving channel portion 212 includes a top slot portion 214 and first and second side tongue members 216A, 216B extending along the length thereof. In addition, a circular channel 218 is provided on the top of housing portion 218.

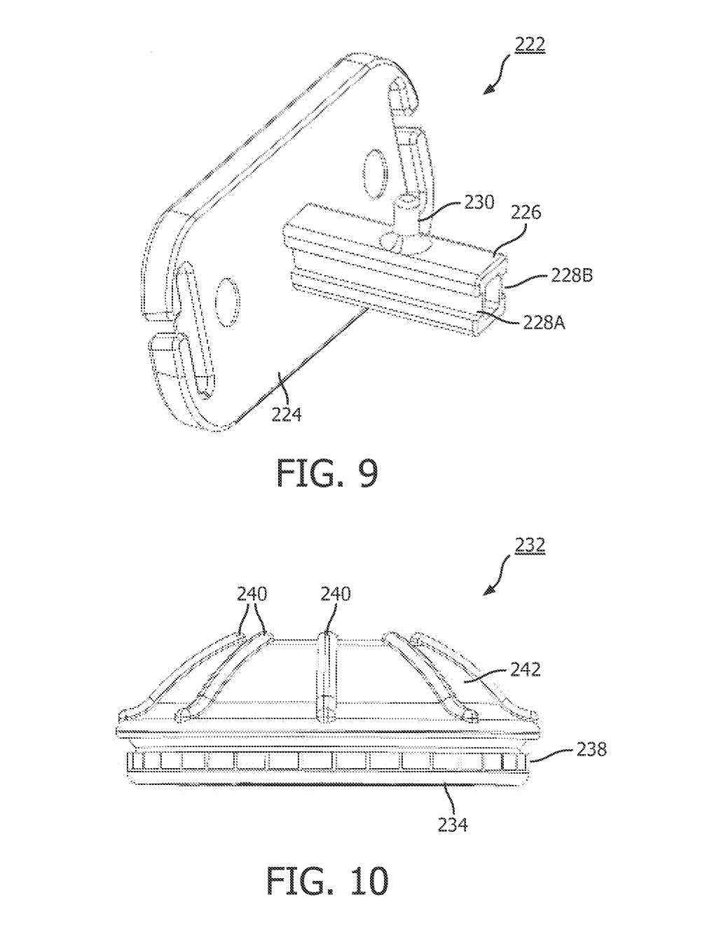

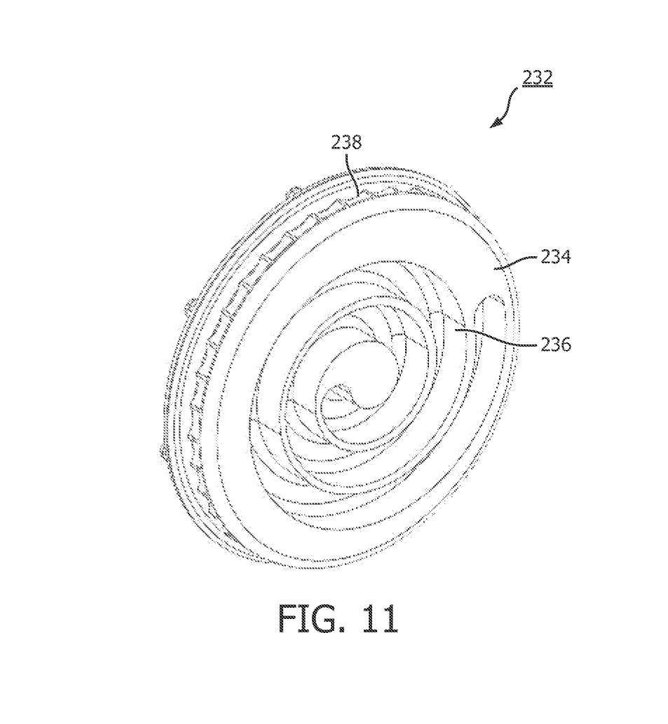

Adjustment mechanism 202 also includes a linear translation assembly 220 partially received and housed within housing portion 206. Linear translation assembly 220 includes a forehead cushion support member 222, shown in FIG. 9, and a rotatable knob member 232, shown in FIGS. 10 and 11. Forehead cushion support member 222 includes a base portion 224 to which forehead cushion 204 is attached, and an elongated post member 226 extending from base portion 224 and having first and second side grooves 228A, 228B formed therein. In an alternative embodiment, tongue members 216A, 216B may be provided on elongated post member 226 and grooves 228A, 228B may be provided in receiving channel 212. In addition, a post 230 is provided on the top surface of elongated post member 226.

As seen in FIGS. 10 and 11, rotatable knob member 232 is in the exemplary embodiment a dome shaped member having a bottom surface 234 having a helical groove 236 provided therein that is structured to receive post 230. In addition, a bottom lip 238 extends around the outer circumference of knob member 232. A plurality of ridges 240 are provided on the top surface 242 of knob member 232.

Adjustment mechanism 202 is assembled by inserting the front end of elongated post member 226 into receiving channel portion 212 of housing portion 206 in a manner wherein tongue members 216A, 216B are received in grooves 228A, 228B. In addition, when this is done, post 230 will extend through top slot portion 214. Knob member 232 is then coupled thereto in a manner wherein post 230 is inserted into helical groove 236 and bottom lip 238 is received within circular channel 218. By rotating knob member 232, the position of post 230 along helical groove 236 will be caused to change, which in turn will cause the position of housing portion 206 and frame member 208 relative to forehead cushion support member 222 and forehead cushion 204 (which will be at a fixed position on the patient's head) to be linearly adjusted, thereby allowing for selective adjustment of the force that is applied to the bridge of the patient's nose as described elsewhere herein.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A patient interface device, comprising:
a patient sealing assembly for delivering a flow of breathing gas to an airway of a patient, the patient sealing assembly including a cushion and a frame member coupled to the cushion the frame member having an elongated portion extending away from the cushion in a first direction; and
an adjustable forehead support assembly provided at a distal end of the elongated portion of the frame member, the adjustable forehead support assembly including an adjustment mechanism coupled to a forehead cushion, the adjustment mechanism including a forehead cushion support member having a base portion coupled to the forehead cushion, a projecting member extending from the base portion in a second direction generally perpendicular to the first direction, a rotatable knob member having a surface having a helical groove provided therein, the rotatable knob member being rotatable about an axis extending in the first direction and within a plane parallel to the second direction, and a post extending from one of the frame member and the projecting member in the first direction, wherein the post is received within the helical groove, wherein rotation of the knob member causes a position of the post within the helical groove to change thereby causing relative linear movement between the projecting member and the frame member in the second direction, and wherein linear movement of the frame member relative to the projecting member causes movement of the frame member and the cushion relative to the forehead cushion.

2. The patient interface device according to claim 1, wherein the post is coupled to the projecting member, wherein the adjustment mechanism includes a housing coupled to the frame member, and wherein the projecting member and the knob member are each received within the housing.

3. The patient interface device according to claim 2, wherein the rotatable knob member includes a bottom lip extending around an outer circumference of the knob member, wherein the housing includes a circular channel, and wherein the bottom lip is received within the circular channel.

4. The patient interface device according to claim 2, wherein the housing includes a receiving channel portion, wherein the projecting member is moveably received within the receiving channel portion, and wherein one of the projecting member and the receiving channel portion includes a tongue member and the other of the elongated post member and the receiving channel portion includes a groove that mates with the tongue member.

5. The patient interface device according to claim 2, wherein the housing includes a receiving channel portion having a slot portion, wherein the projecting member is moveably received within the receiving channel portion in a manner wherein the post extends through the slot portion.

6. The patient interface device according to claim 1, wherein the post is coupled to a drive beam attached to the frame member, and wherein the knob member is rotatably coupled to the projecting member.

7. The patient interface device according to claim 6, wherein the projecting member includes a first support member having an orifice provided therein and a second support member, wherein a gap is provided in between the first support member and the second support member, wherein the post is provided on a distal end of the first support member, wherein the drive beam is received within the gap and wherein the post extends through the orifice.

8. A method of adjusting a patient interface device having a patient sealing assembly for delivering a flow of breathing gas to an airway of a patient, the patient sealing assembly including a cushion and a frame member coupled to the cushion, the frame member having an elongated portion extending away from the cushion in a first direction, and an adjustable forehead support assembly provided at a distal end of the elongated portion of the frame member, the adjustable forehead support assembly including a forehead cushion, a forehead cushion support member coupled to the forehead cushion, and a rotatable knob member having a surface having a helical groove provided therein that is operatively coupled to the forehead cushion support member, the rotatable knob member being rotatable about an axis extending in the first direction and within a plane generally perpendicular to the first direction, the method comprising:
rotating the knob member in a first rotation direction and thereby causing the frame member to move in a first direction toward the forehead cushion; and
rotating the knob member in a second rotation direction and thereby causing the frame member to move in a second direction away from the forehead cushion.

9. The method according to claim 8, wherein the helical groove receives a post extending from one of the frame member and the forehead cushion support member in the first direction, wherein rotation of the knob member causes a position of the post within the helical grove to change thereby causing relative linear movement between the forehead cushion support member and the frame member in a second direction perpendicular to the first direction.
